(19) United States
(12) Patent Application Publication
(10) Pub. No.: US 2020/0368371 A1
(43) Pub. Date: Nov. 26, 2020

(54) DYE-PROTEIN COMPLEX FOR NIR II AND PHOTOACOUSTIC IMAGING

(71) Applicant: The Board of Trustees of the Leland Stanford Junior University, Stanford, CA (US)

(72) Inventors: Zhen Cheng, Mountain View, CA (US); Bulin Du, Palo Alto, CA (US)

(21) Appl. No.: 16/879,851

(22) Filed: May 21, 2020

Related U.S. Application Data
(60) Provisional application No. 62/851,732, filed on May 23, 2019.

(51) Int. Cl. A61K 49/00

(52) U.S. Cl. CPC A61K 49/0056; A61K 49/0032

(57) ABSTRACT

Provided are embodiments of a probe, methods of manufacture, and use in photoacoustic and NIR-II near-infrared imaging, the probe comprising a fluorescent dye and a human serum albumin molecule, or fragment thereof, wherein the fluorescent dye is attached to the human serum albumin molecule, and wherein the fluorescent dye can have a greater near-infrared fluorescence intensity than when not in contact with the human serum albumin molecule.

### CROSS-REFERENCE TO RELATED APPLICATIONS

This application claims priority to U.S. Provisional Patent Application Ser. No. 62/851,732 entitled “IR-820 DYE-PROTEIN COMPLEX FOR NIR HAND PHOTOACOUSTIC IMAGING” filed on May 23, 2019, the entirety of which is hereby incorporated by reference.

### STATEMENT REGARDING FEDERALLY SPONSORED RESEARCH

This invention was made with government support under grant number DE-SC0008397 awarded by the United States Department of Energy. The government has certain rights in the invention.

### BACKGROUND

Because of reduced scattering, minimal absorption and negligible auto fluorescence, the second near-infrared biological window (NIR-II, 1000-1700 nm) imaging provides high resolution, high signal-to-noise ratio, and deep tissue penetration capability (He et al., (2018) Chem. Soc. Rev. 47: 4258-4278). Several types of inorganic and organic NIR-II fluorophores have been reported. Inorganic NIR-II fluorophores, including single-walled carbon nanotubes (SWCNTs), quantum dots (QDs), and rare earth-doped nanoparticles (RENPs) exhibit advantageous optical properties but slow excretion kinetics. Therefore, prolonged retention in the organs of the reticuloendothelial system raise critical safety concerns for clinical translation (Ding et al., (2019) Adv. Healthc. Mater. (2019) e1900260; He et al., (2019) Nano Lett.). Organic NIR-II dyes offer distinct advantages with regard to clinical translation. The development of organic small-molecule fluorophores represents an important, newly emerging and dynamic field in molecular imaging (He et al., (2018) Chem. Soc. Rev. 47: 4258-4278; Li et al., (2018) Angew. Chem. Int 57: 7483-7487; Antaris et al., (2017) Nat. Commun. 8 15269). Organic fluorophores, based on donor-acceptor-donor (D-A-D) structure with benzobisthiadiazole derivatives as the acceptor, exhibit NIR-II emission spectra in organic solvents (Antaris et al., (2016) Nat. Mater. 15: 235-242; Shou et al., (2017) Adv. Funct. Mater. 27; Sun et al., (2017) Chem. Sci. 8: 3489-3493). A polymethine fluorophore having a π structure can also drive the wavelength over 1000 nm (Ding et al., (2019) J. Med. Chem. 62 (2019) 2049-2059; Lei et al., (2019) Angew. Chem.). However, the disadvantage of complicated synthesis steps, low solubility in aqueous solution or poor quantum yield remain challenges for their clinical translation.

After the clinically-approved cyanine fluorophores such as indocyanine green (ICG) exhibiting a significant amount of NIR-II emission was first reported (Antaris et al., (2017) Nat. Commun. 8 15269), spectroscopic characterization of commercially available NIR-I dye revealed long emission tails that stretch into the NIR-II region over 1000 nm. This opened a new route to clinical NIR-II imaging (Zhu et al., (2018) Theranostics 8: 4141-4151) that enables NIR-II imaging with direct translation potential into clinical settings, and even outperforms other commercially-available NIR-II emitters (Carr et al., (2018) Proc. Natl. Acad. Sci. U.S.A. 115: 4465-4470; Starosolski et al., (2017) PLoS ONE 12: e0187563; Zhu et al., (2018) Bioimaging Adv. Mater. e1802546).

Cyanine dye IR-820, one of the commercially available NIR-I dyes, has similar optical and thermal generation properties to ICG, but has improved stability and longer plasma half-life in vitro and in vivo (Fernandez-Fernandez et al., (2012) Mol. Imaging 11: 7290; Prajapati et al., (2009) Mol. Imaging 8: 45-54). The chemical structure of IR-820 is comparable to that of ICG, with the presence of a chlorobenzene ring that can increase molecular stability (Fernandez-Fernandez et al., (2012) Mol. Imaging 11: 7290). IR-820 has been conjugated with polyethyleneimine to delivery DNA, which can be monitored in vivo with noninvasive optical imaging (Masotti et al., (2008) Bioconjug. Chem. 19: 983-987).

### SUMMARY

One aspect of the disclosure, therefore, encompasses embodiments of a probe comprising a fluorescent dye and a human serum albumin molecule, or fragment thereof, wherein the fluorescent dye is attached to the human serum albumin molecule, and wherein the fluorescent dye can have a greater near-infrared fluorescence intensity than when not in contact with the human serum albumin molecule.

In some embodiments of this aspect of the disclosure, the fluorescent dye can also be a photoacoustic emitter.

In some embodiments of this aspect of the disclosure, the fluorescent dye can be a cyanine dye.

In some embodiments of this aspect of the disclosure, the fluorescent dye can be IR-820.

In some embodiments of this aspect of the disclosure, the dye can be attached to the human serum albumin, or fragment thereof, by at least one electrostatic bond, at least one covalent bond, or a combination thereof.

In some embodiments of this aspect of the disclosure, the dye can be conjugated to the human serum albumin, or fragment thereof, by at least one covalent bond.

In some embodiments of this aspect of the disclosure, the probe can be admixed with a pharmaceutically acceptable carrier.

Another aspect of the disclosure encompasses embodiments of a method of enhancing the NIR-II emission of a fluorescent dye, the method comprising synthesizing a composition comprising a fluorescent dye attached to a human serum albumin molecule, or fragment thereof, and wherein the fluorescent dye can have a greater near-infrared fluorescence than when not in contact with the human serum albumin molecule, the method comprising mixing aqueous solutions of the fluorescent dye and the human serum albumin, or fragment thereof, wherein the fluorescent dye and the human serum albumin, or fragment thereof, can have a molar ratio selected from the range of about 10:1 to about 1:10.

In some embodiments of this aspect of the disclosure, the fluorescent dye and the human serum albumin, or fragment thereof, can have a molar ratio of about 1:1.

In some embodiments of this aspect of the disclosure, the fluorescent dye can be a cyanine dye.

In some embodiments of this aspect of the disclosure, the fluorescent dye can be IR-820.

Still another aspect of the disclosure encompasses embodiments of a method of imaging a tissue or organ in an animal or human subject, the method comprising the steps of administering to an animal or human subject an amount of a probe, wherein the probe can comprise a fluorescent dye attached to a human serum albumin molecule, or fragment thereof, and wherein the fluorescent dye has a greater near-infrared fluorescence intensity than when not in contact with the human serum albumin molecule, generating a fluorescent signal from the probe by irradiating the exterior surface or a tissue of the subject with an excitation radiation specific for the fluorescent dye, detecting the emitted fluorescence and generating an image of the fluorescence relative to the body of the subject.

In some embodiments of this aspect of the disclosure, the method can further comprise irradiating the subject with a laser energy capable of generating a photoacoustic signal from the administered probe, detecting the photoacoustic signal emitted by the irradiated probe and generating an image of the fluorescence relative to the body of the subject.

In some embodiments of this aspect of the disclosure, the probe can be concentrated in a tumor of the subject, and wherein the photoacoustic energy generated from the probe can reduce at least one of the proliferation or the viability of the tumor.

### BRIEF DESCRIPTION OF THE DRAWINGS

Many aspects of the disclosure can be better understood with reference to the following drawings.

FIGS. 1A-1C illustrate the molecular modeling of the interaction of IR-820 with HSA. FIG. 1A shows the different subdomains of the HSA protein (in ribbon).

FIG. 1B illustrates the electronic potential mapping of IR-820 docked on HSA molecule. The protein electronic potential becomes more positive.

FIG. 10 illustrates the docking site around IR-820 molecule (presented as sticks).

FIGS. 2A-2C illustrate the spectral characterization and stability of IR-820-HSA.

FIG. 2A shows a NIR-II fluorescent image (10 ms, 1000LP) of IR-820, IR-820-HSA molar ratio of 1:2, and IR-820-HSA heated at 60° C. for 10 mins in deionized water with a constant IR-820 concentration of 10 μM.

FIG. 2B shows the absorbance of IR-820, IR-820-HSA, and IR-820-HSA heated in deionized water.

FIG. 2C shows the fluorescent emission spectra of IR-820, IR-820-HSA and IR-820-HSA heated in deionized water after excitation at 808 nm laser.

FIGS. 3A-3E illustrate NIR-II imaging of vascular system.

FIGS. 3A-3C illustrate NIR II images (3000 ms, 1150LP) obtained 30 mins after intravenous injection of IR-820-HSA into C57BL/6 mice.

FIG. 3D illustrates SBRs (white and black dash lines in FIGS. 3B and 3C) analyzed for right hind limb and abdomen vessels based on the cross-sectional intensity profiles.

FIG. 3E illustrates FWHM (white and black dash lines in FIGS. 3B and 3C) analyzed for right hind limb and abdomen vessels based on the cross-sectional intensity profiles.

FIGS. 4A-4F illustrate NIR-II imaging of lymphatic system.

FIG. 4A shows the popliteal lymph node (white arrowheads) and sciatic lymph node (dark arrowheads) connected with lymphatic vessels were easily identified after probe injected from footpad in each side in prone position (1000 ms, 1000LP).

FIGS. 4B and 4C illustrate right inguinal lymph node (dark arrowheads) and afferent and efferent lymphatic vessels shown after probe was injected from tail in lateral position and supine position (1000 ms, 1000LP).

FIGS. 4D-4F are graphs showing FWHM and SBRs basing on the cross-sectional intensity profiles of lymphatic vessels marked by a white and black dash line (marked by a white and black dash line in FIG. 4A-4C).

FIGS. 5A-5F illustrate NIR-II imaging for assessment of tumor blood supply network.

FIGS. 5A and 5B show NIR-II fluorescence images captured on the U87 glioma tumor-bearing mouse (after the IR-820-HSA heated was intravenously injection) under 1000LP and 1200LP filters (1000 ms) with mice in the prone position.

FIG. 5C shows FWHM and SBRs based on the cross-sectional intensity profiles of tumor blood vessels (marked by a white and black dash line in FIGS. 5A and 5B).

FIGS. 5D-5F show NIR II images (1000 ms, 1000LP) of the main blood supply arteries at time 0, a week later (white and black arrow) with mice in the supine position, and after dissection.

FIGS. 6A-6E illustrate NIR-II and PA imaging of tumors.

FIG. 6A shows in vivo fluorescence images (300 ms 1000Lp) of 143B osteosarcoma tumor-bearing nude mice taken at different time points post injection of IR-820-HSA.

FIG. 6B shows the in vivo 3D-volume rendering of photoacoustic images (view of left side) of a 143B osteosarcoma tumor taken at different time points post-injection of IR-820-HSA.

FIG. 6C shows the time-signal intensity curve of tumor, background and tumor-to-background ratios based on images showed in FIG. 6A.

FIG. 6D shows ex vivo fluorescence images (300 ms, 1000Lp) of major organs (spleen, liver, tumor, heart, kidney and lung) and tumor (separated from the middle) dissected from 143B tumor-bearing mice 8 h after IR-820-HSA imaging.

FIG. 6E shows semi-quantitative analysis of HSA-IR-820 biodistribution in tumor-bearing mice by measuring fluorescence intensity.

FIG. 7 illustrates IR-II image-guided tumor resection. The series of images show the process of the tumor being excised under NIR-II imaging after IR802-HSA was injected 72 h (1000 ms, 1000LP). The process involved open skin, tumor exposure and complete tumor resection.

FIG. 8 illustrates fluorescence intensity in different concentration of IR-820 in deionized water and phosphate buffer saline.

FIG. 9 shows the fluorescence intensity enhancement of IR-820-HSA complex at different HSA: IR-820 molar ratios.

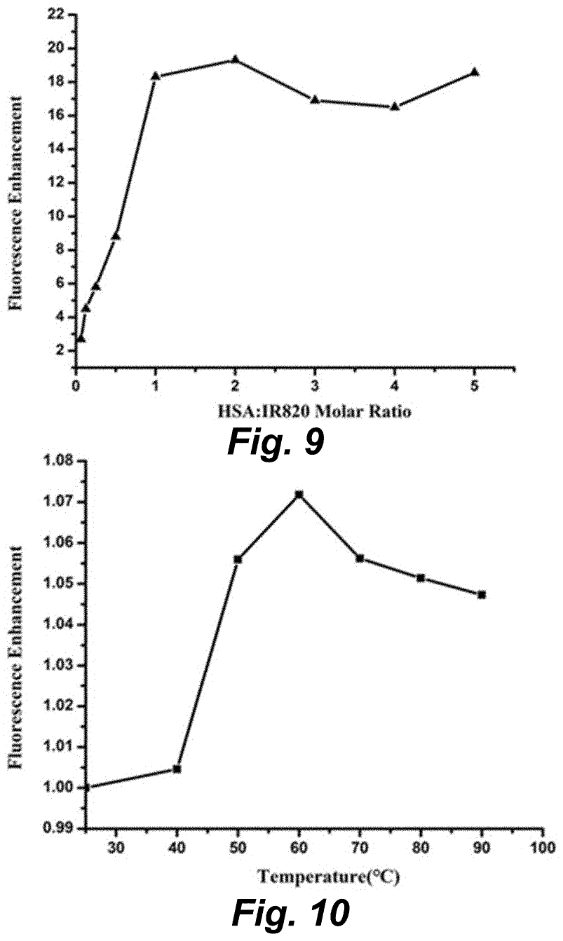

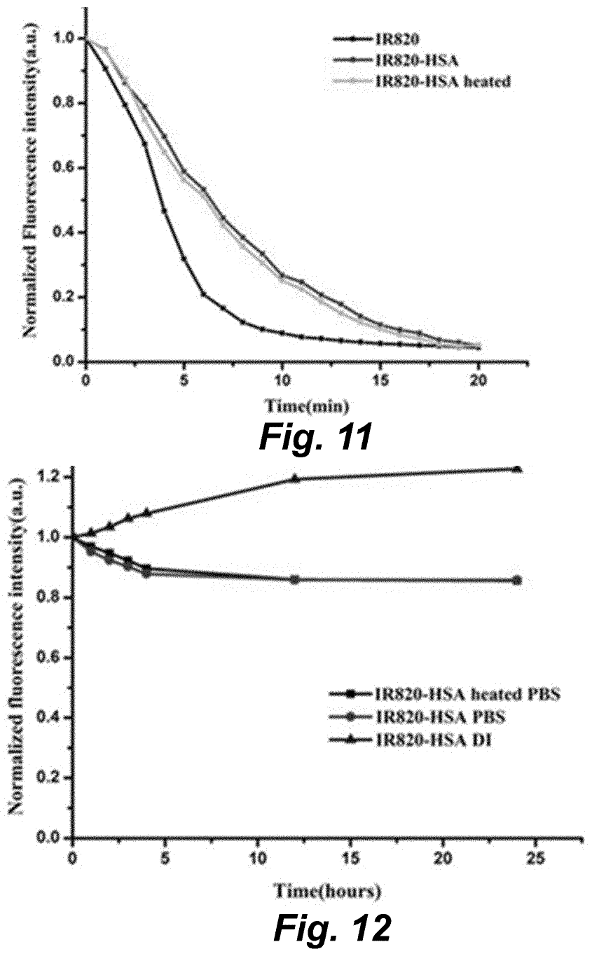

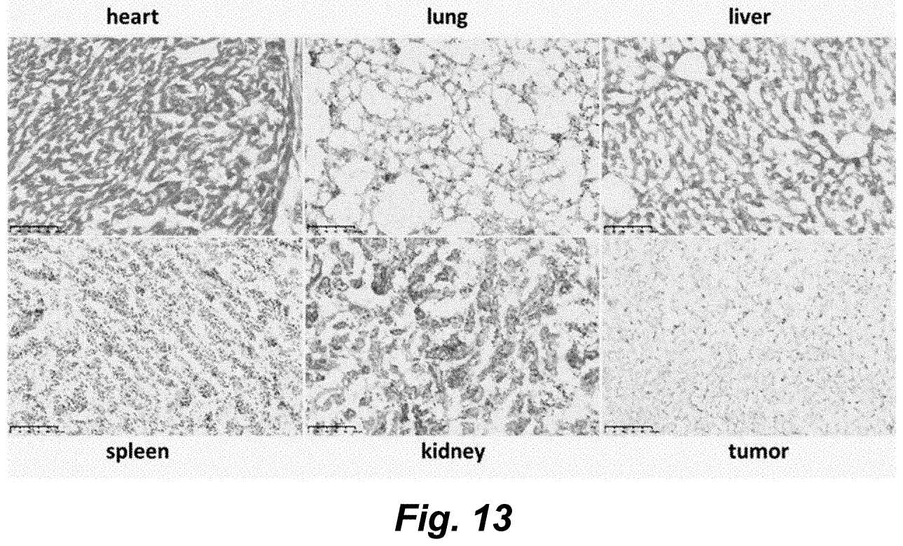

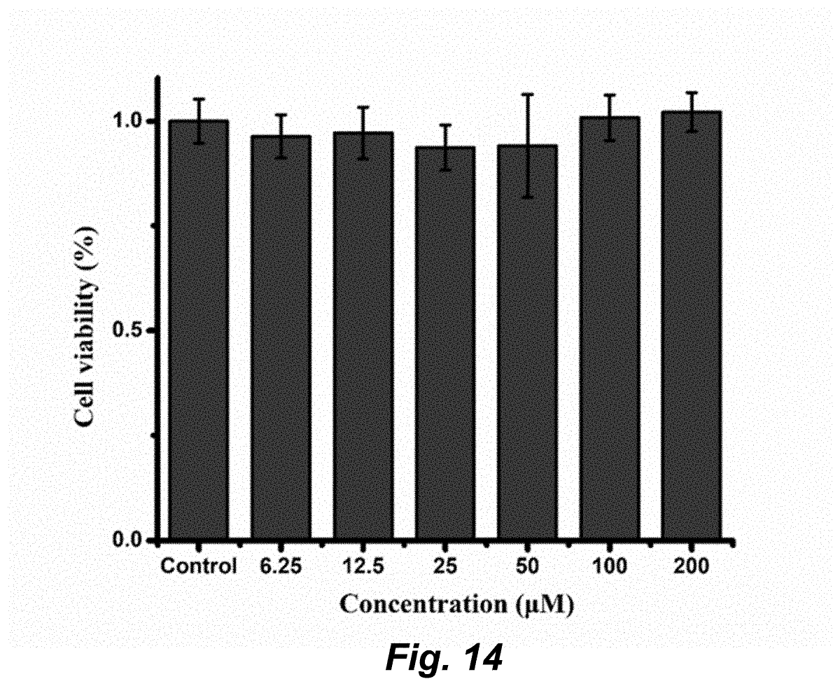

FIG. 10 shows fluorescence intensity enhancement of IR-820-HSA complex after heating at different temperature for 10 mins.

FIGS. 11 and 12 illustrate the stability of IR-820-HSA exposed to continuous illumination and in PBS.

FIG. 11 shows photobleaching curves of IR-820, IR-820-HSA, and the IR-820-HSA heated complex in deionized water exposed to continuous illumination at 808 nm for 25 minutes.

FIG. 12 shows the stability of IR-820-HSA and IR-820-HSA heated in PBS compared with IR-820-HSA in deionized water.

FIG. 13 illustrates H&E stained images of major organ (heart, lung, liver, spleen, kidney) and tumor slices collected from mice with IR-820-HSA injection 7 days previously. No abnormalities were observed.

FIG. 14 illustrates the results of a toxicity assay of IR-820-HSA. MTT analysis of IR-820-HSA in NIH3T3 cells indicated no apparent cytotoxicity to the cell lines and excellent biocompatibility in vitro.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, toxicology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### Definitions

The term “detectable signal emitter”, for the purposes of the specification or claims, means a label molecule that is incorporated indirectly or directly into a nanoparticle, wherein the label molecule facilitates the detection of the nanoparticle in which it is incorporated. Thus, “detectable signal emitter” is used synonymously with “label molecule.”

The term “detectable” refers to the ability to detect a signal over the background signal. The detectable signal is defined as an amount sufficient to yield an acceptable image using equipment that is available for pre-clinical use. A detectable signal maybe generated by one or more administrations of the probes of the present disclosure. The amount administered can vary according to factors such as the degree of susceptibility of the individual, the age, sex, and weight of the individual, idiosyncratic responses of the individual, the dosimetry, and the like. The amount administered can also vary according to instrument and digital processing related factors.

The term “in vivo imaging” as used herein refers to methods or processes in which the structural, functional, or physiological state of a living being is examinable without the need for a life-ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “optical energy” as used herein refers to electromagnetic radiation between the wavelengths of about 350 nm to about 800 nm and which can be absorbed by the dyes or cellulose-based nanoparticles of the embodiments of the photoacoustic probes of the disclosure. The term “optical energy” may be construed to include laser light energy or non-laser energy.

The term “detectable imaging moiety” or “label” as used herein refers to an inorganic or organic molecule that may be detected by an optical method, for example by fluorescence detection, light absorbance and the like. It should be noted that reference to detecting a signal from a probe also includes detecting a signal from a plurality of probes. In some embodiments, a signal may only be detected that is produced by a plurality of probes. Additional details regarding detecting signals (e.g., acoustic signals) are described below.

The “imaging moiety” may be detected either externally to a subject human or non-human animal body or via use of detectors designed for use in vivo, such as optical detectors such as endoscopes. The imaging moiety is preferably a reporter suitable for in vivo optical imaging. The term “imaging moiety” as used herein may further refer to a reporter suitable for in vivo optical imaging and the reporter is any moiety capable of detection either directly or indirectly in an optical imaging procedure. The reporter can be a light scatterer (e.g. a colored or uncolored particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most advantageously, the reporter has fluorescent properties.

Organic chromophoric and fluorophoric reporters suitable for use in the probes of the disclosure include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoaniline dyes, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes.

Particular examples of chromophores which may be used include: fluorescein, sulforhodamine 101 (Texas Red), rhodamine B, rhodamine 6G, rhodamine 19, indocyanine green, Cy2, Cy3, Cy3B, Cy3.5, Cy5, Cy5.5, Cy7, Cy7.5, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750.

Particularly advantageous are dyes which have absorption maxima in the visible or near infrared (NIR) region, between 400 nm and 3 μm, particularly between 600 and 1300 nm. Optical imaging modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching.

The term “fluorophore” as used herein refers to a component of a molecule that causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Fluorophores for use in the compositions of the disclosure include, but are not limited to, fluorescein isothiocyanate (FITC), a reactive derivative of fluorescein, which has been one of the most common fluorophores chemically attached to other, non-fluorescent, and molecules to create new fluorescent molecules for a variety of applications. Other historically common fluorophores are derivatives of rhodamine (TRITC), coumarin, and cyanine, such as, but not limited to, indocyanine green IR-820. Newer generations of fluorophores such as the ALEXA FLUORS® and the DYLIGHT FLUORS® are generally more photostable, brighter, and less pH-sensitive than other standard dyes of comparable excitation and emission.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

### DESCRIPTION

Much attention has been paid to the development of second near infrared (NIR-II) fluorescence imaging because of reduced scattering, minimal absorption and negligible auto fluorescence. With emission in the window of 1000-1700 nm, NIR-II bioimaging allows visualization of deep anatomical features with an unprecedented degree of clarity. In addition to development of new NIR-II agents, NIR-II imaging via non-peak NIR-I fluorescence emission over 1000 nm is becoming promising, especially for commercially available NIR-I dyes.

It was demonstrated that changing the functional groups of CH1055 from carboxylic to sulfonic acid results in a readily formed supramolecular assembly with plasma proteins, which induced a brilliant increase in fluorescent brightness (Antaris et al., (2017) Nat. Commun. 8 15269). Human serum albumin (HSA), as an abundant plasma protein in blood and tissue fluids, can attach with molecular probes covalently or non-covalently, HSA-based probe design, therefore, represents an advantageous strategy for developing molecular probes for theranostics of diseases, especially for tumors with natural passive albumin accumulation (Hoogenboezem & Duvall (2018) Adv. Drug Deliv. Rev. 130: 73-89; Chen et al., (2014) Biomaterials 35: 9355-9362; Chen et al., (2014) Biomaterials 35: 8206-8214). The present disclosure provides a dye-protein complex, including an IR-820-HSA organic complex, to demonstrate a fluorescent increase and its use for NIR-II imaging. Meanwhile, photoacoustic imaging (PAI) in the NIR-II window offers numerous advantages, including high spatial resolution, deeper penetration depth, reduced optical absorption, and tissue scattering (Upputuri & Pramanik, (2019) J. Biomed. Opt. 24: 1-20; He et al., (2019) Advanced Optical Materials 110 (2019) 1900045).

The present disclosure encompasses embodiments of supramolecular assemblies of protein complexes with a sulfonated NIR-I organic dye (IR-820) to produce a significant increase in fluorescence for NIR-II imaging. In vivo NIR-II imaging with IR-820-HSA can non-invasively and dynamically visualize and monitor the physiological and pathological conditions of the vascular system, lymphatic drainage system, tumor-bearing mice, and even image-guided tumor resection with high spatial and temporal resolution.

### Preparation of IR-820-HSA Complex:

IR-820 was first dissolved in deionized water, and then added to deionized water or PBS to determine the optimal concentration. FIG. 8 illustrates 10 μM in deionized water with the highest fluorescence intensity in designed concentration but not in PBS for poor solubility or severe quenching. 10 μM was determined as the concentration for other studies. To develop a brightness NIR-II probe by exploiting IR-820-HSA interactions, HSA was added to IR-820 to observe the increase in fluorescence enhancement under the NIR-II camera. Different HSA: IR-820 ratios were prepared while keeping IR-820 at a constant 10 μM to determine the approximate binding stoichiometry.

As seen in FIG. 9, the maximum fluorescence intensity occurred at molar ratio of 1:2 with about a 19-fold fluorescence enhancement. FIG. 10 shows the brightness slightly increased about 1.07-fold with an increase in temperature for 10 mins until 60° C. was attained. Heating of the complex was attempted with the hope of exposing IR-820 to typically inaccessible hydrophobic domains located in the protein interior, and speeding up the binding process.

The brightness of NIR-I dyes, such as IR-12N3, ICG, and IRDye800, in bovine serum albumin (BSA), fetal bovine serum (FBS), and PBS was determined as a function of temperature. The optimal condition with IR12-N3-FBS demonstrated a fluorescence enhancement factor around 6-fold (Zhu et al., (2018) Bioimaging Adv. Mater. e1802546). The IR-820-HSA complex of the present disclosure showed a notable brightness enhancement of 20-fold.

### Molecular Modeling:

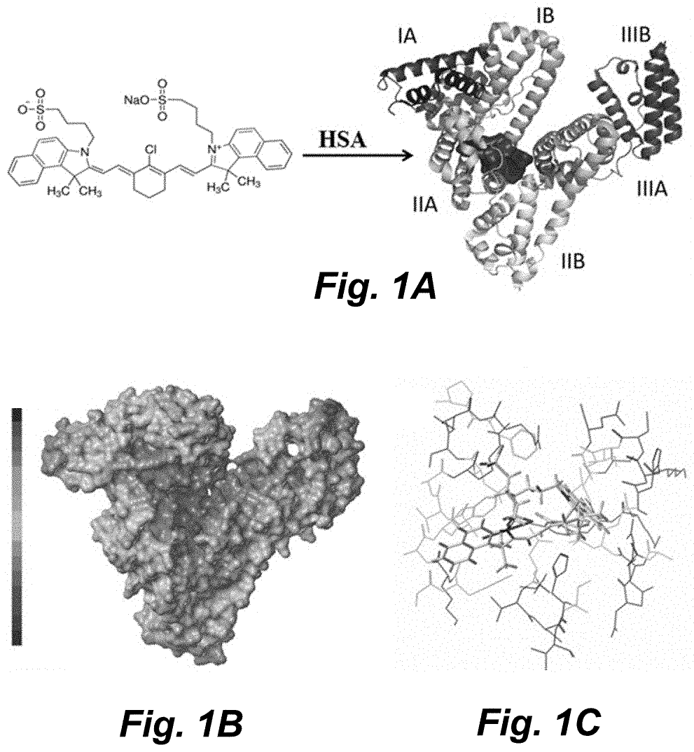

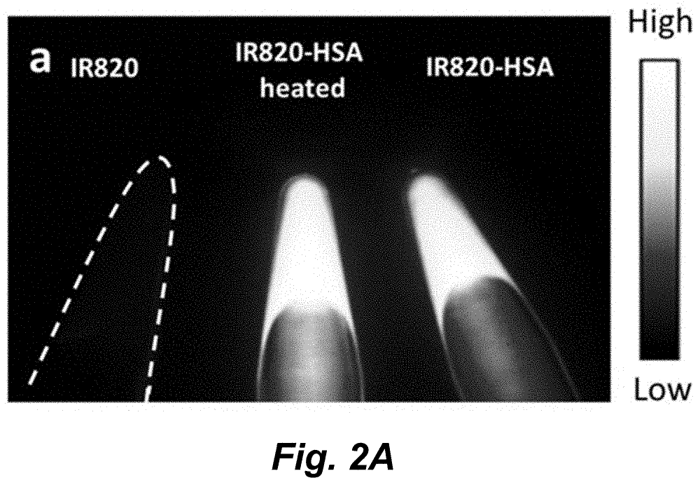

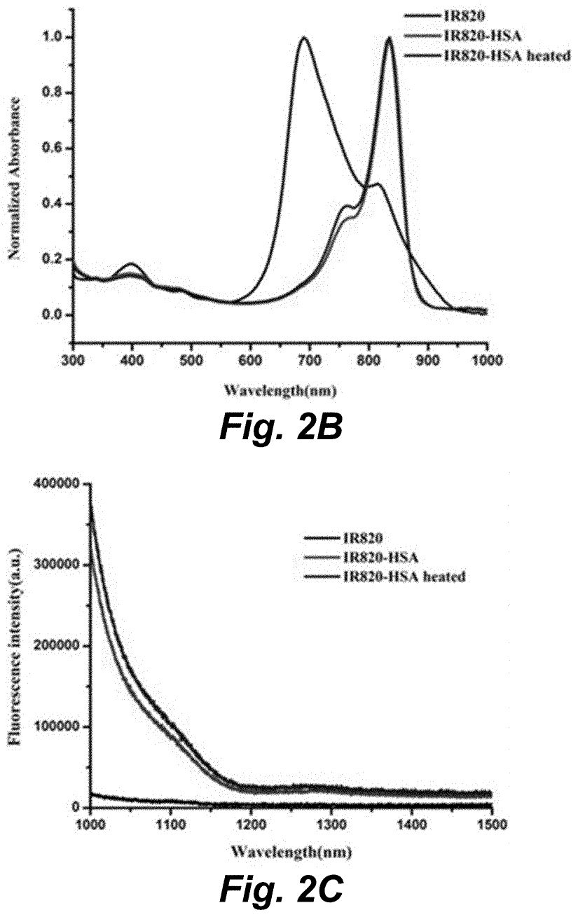

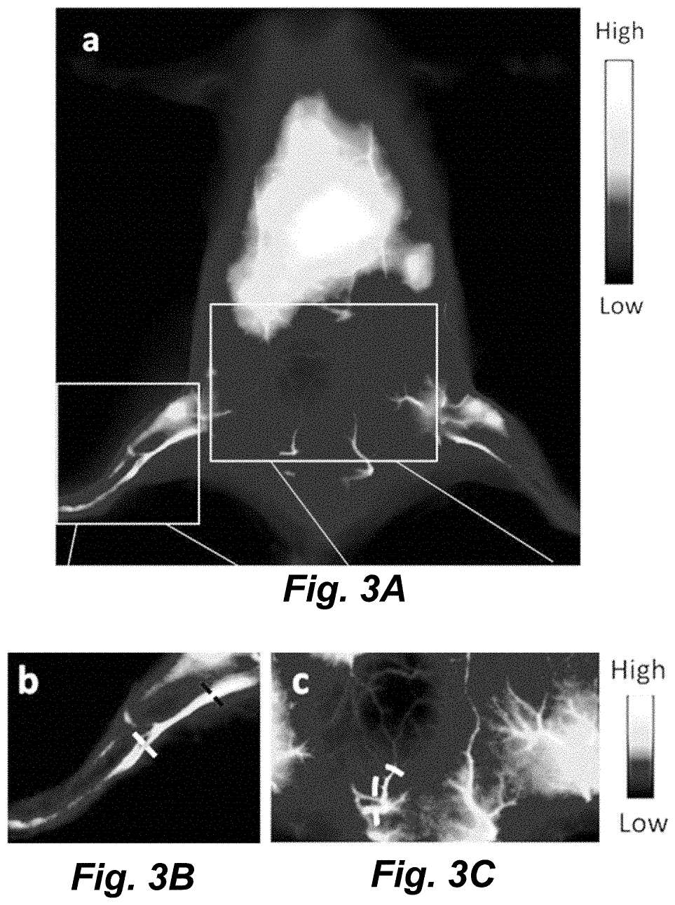

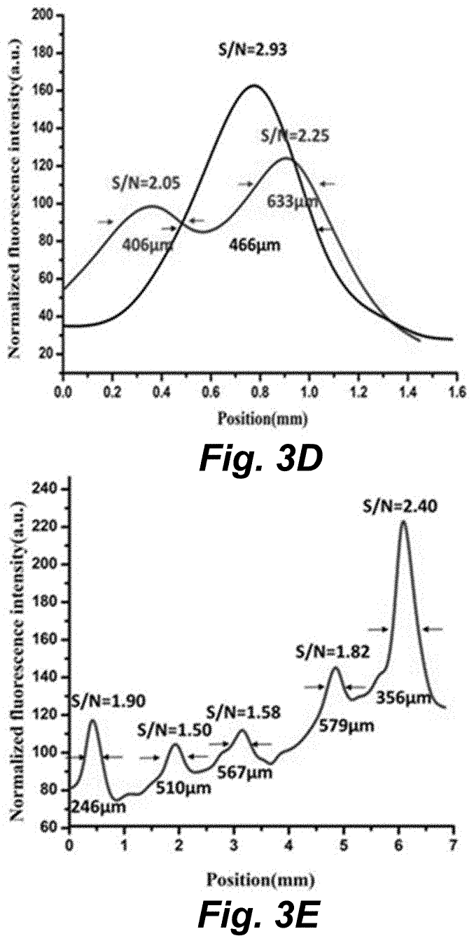

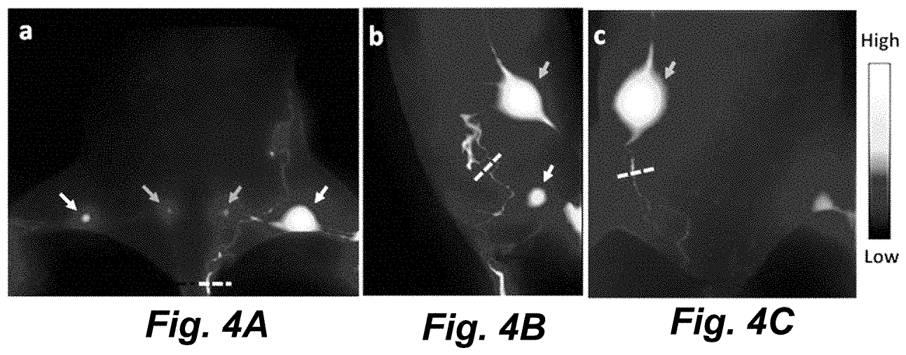

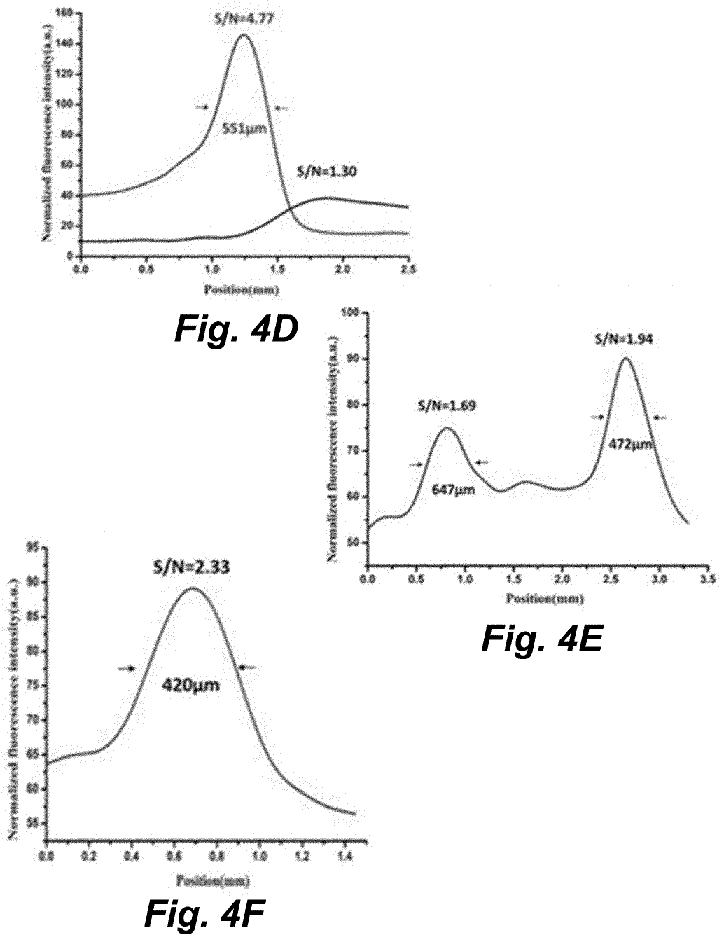

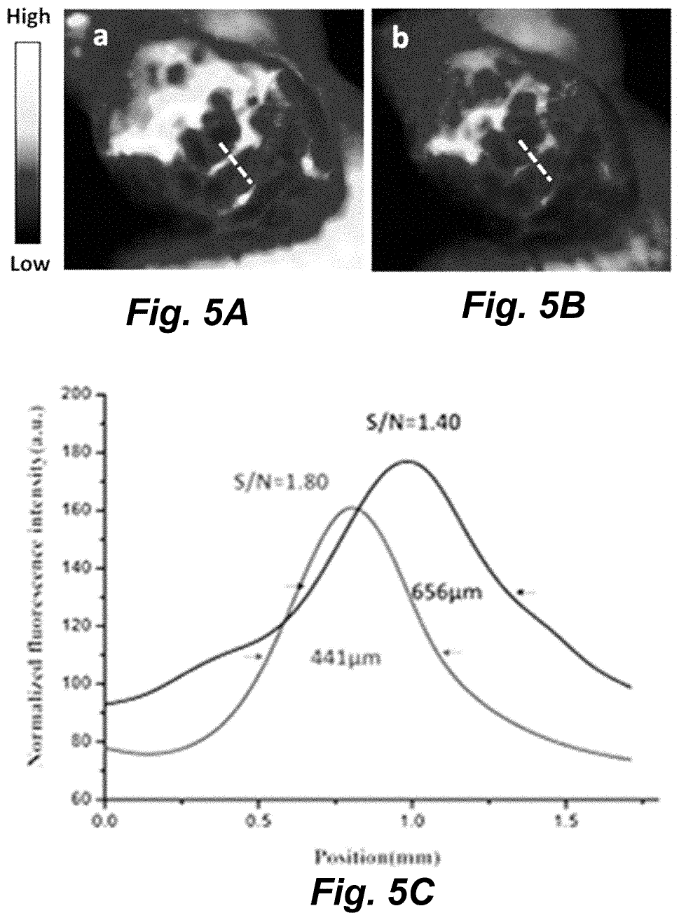

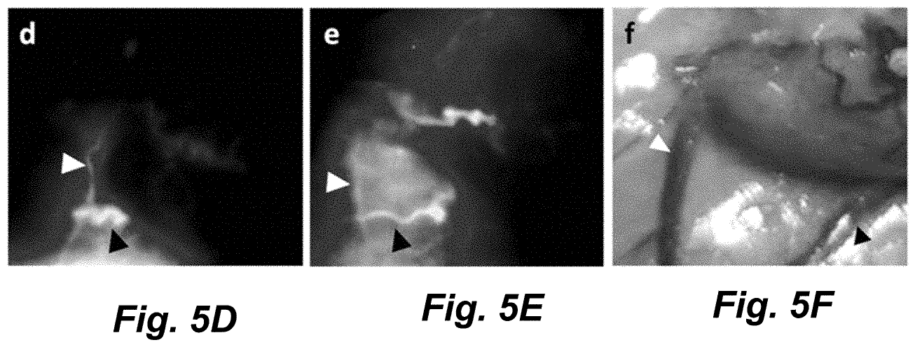

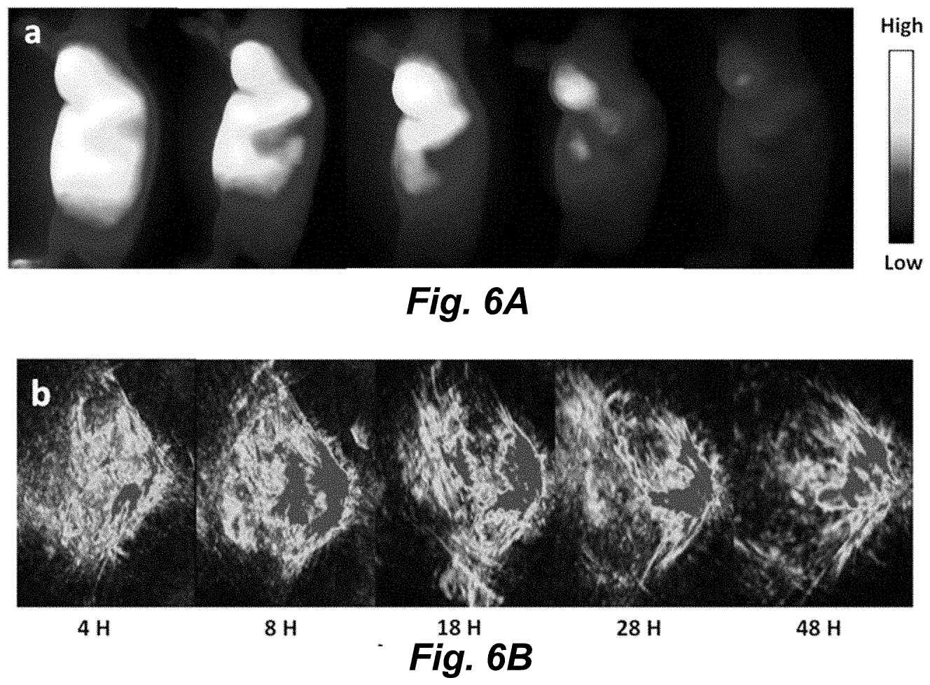

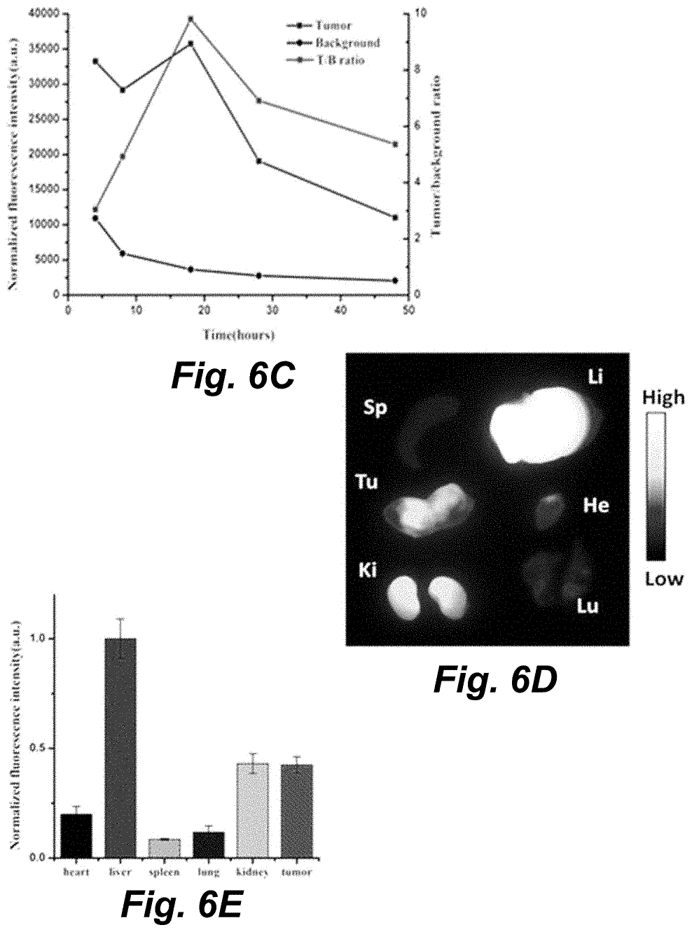

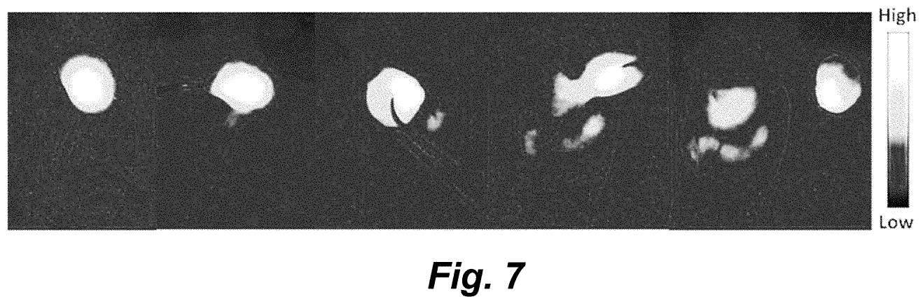

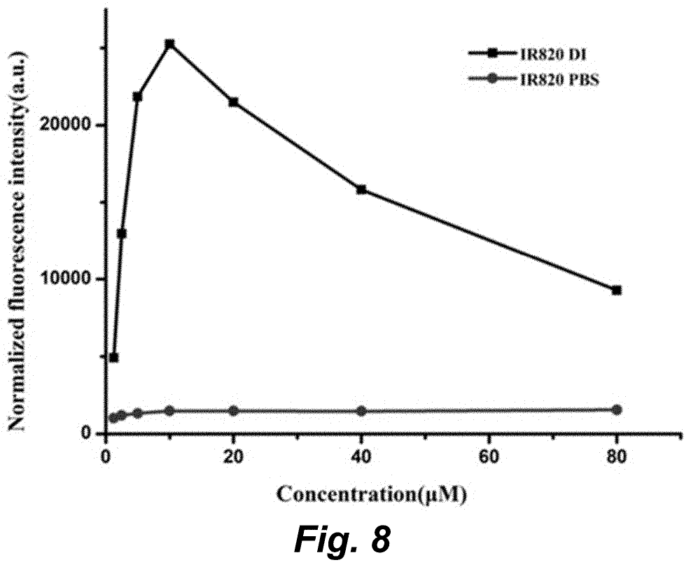

To investigate the binding site of IR-820 with HSA molecular modeling using SybyI-X software was used. From the docking results in FIG. 1A it is apparent that the IR-820 is located in sub-domain IIA of the HSA. Many commonly used drugs with acidic or electronegative features bind to HSA, usually at one of two primary sites, located in subdomains IIA and IIIA, respectively (Hoogenboezem & Duvall (2018) Adv. Drug Deliv. Rev. 130: 73-89; Ghuman et al., (2005) J. Mol. Biol. 353: 38-52). As shown in FIG. 1B, subdomain IIA is negatively charged, and surrounded by also negatively charged outer surface, which will facilitate the combination of HSA and IR-820. IIA also contains a large hydrophobic cavity with the ability to bind many kinds of drugs, which is in consist with the result shown in FIG. 10. Ligands binding to site IIA are often aromatic carboxylic acids with a negatively charged acidic group at one end of the molecule away from a hydrophobic center (Hoogenboezem & Duvall (2018) Adv. Drug Deliv. Rev. 130: 73-89; Kragh-Hansen et al., (2002) Biol. Pharm. Bull. 25: 695-704). While not wishing to be bound by any one theory, for IR-820 with negatively charged sulfonic acid group, electrostatic interactions may be the main driving forces for the binding interaction between IR-820 and HSA sub-domain IIA. Upon binding to HSA, the free single-button rotation of IR-820 is limited by HSA, resulting in enhanced fluorescence by a mechanism similar to aggregation-induced emission, in which the free movement of the fluorophore is reduced to produce brighter fluorescence and reduced fluorescence self-quenching (Li & Li (2017) Adv. Sci. (Weinh) 4: 1600484; Anger et al., (2006) Phys. Rev. Lett. 96: 113002). Spectral characterization and stability of IR-820-HSA: As shown in FIG. 2A, IR-820-HSA and heated IR-820-HSA show bright fluorescence enhancement compared to IR-820 in NIR-II fluorescent imaging with a 10 ms exposure time and a 1000 long pass (LP) filter. The absorption curve and fluorescent emission spectra of IR-820, IR-820-HSA and IR-820-HSA heated, all in DI water, are shown in FIGS. 2B and 2C. The free IR-820 dye exhibited a strong peak at 690 nm and a weak shoulder at 815 nm in DI water. After the dye was loaded onto HSA or HSA heated at 60° C. for 10 minutes, the complex exhibited a weak shoulder at 765 nm and a strong peak at 835 nm, with redshifts of 75 nm and 25 nm, respectively. The remarkably redshifted absorption may be due to the strong charge interaction between IR-820 and HSA (Chen et al., (2014) Biomaterials 35: 8206). The absorption curve was similar to the curve of IR-820 dissolved in organic solvents (Fernandez-Fernandez et al., (2012) Mol. Imaging 11: 99). According to the fluorescence emission spectra, IR-820-HSA showed strong fluorescence emission at wavelengths beyond 1000 nm, especially compared to free IR-820.

Previous research demonstrated that IR-820 had greater stability than ICG in aqueous solution under all light conditions (Fernandez-Fernandez et al., (2012) Mol. Imaging 11: 99). It was found that the IR-820-HSA complex in DI water exhibited greater photostability at 808 nm than that of free IR-820 (FIG. 11). The IR-820-HSA complex showed slower photobleaching than free IR-820. Moreover, the photostability of IR-820-HSA was examined in DI water and PBS. As shown in FIG. 12, the fluorescence intensity of IR-820-HSA in PBS was reduced by 15%, and the fluorescence intensity of IR-820-HSA in DI water or FBS was increased nearly by 20%. The enhancement in FBS might be attributable to the increased albumin from FBS. Additionally, the decrease in fluorescence intensity in PBS might be due to an increase in ionic strength in PBS, which partly affects the binding stability between IR-820 and HSA (Jones et al., (2012) Thermochim. Acta 545: 112-115). These findings suggested that IR-820-HSA has high photostability and a suitable absorption and emission tail in the NIR-II region for in vivo imaging.

To explore the potential toxicity of IR-820-HSA in vivo, the mouse embryonic fibroblast cell line NIH3T3 was evaluated in a standard MTT analysis. No apparent cytotoxicity of IR-820-HSA was observed in the cell line even at concentrations up to 200 μM, indicating its low cytotoxicity and excellent biocompatibility in vitro (FIG. 14).

### NIR-II Imaging of Vascular System:

To explore the potential application of IR-820-HSA for reliable assessment of the vascular system in a living subject, heated IR-820-HSA was injected intravenously into C57BL/6 mice through the tail. As shown in FIG. 3A, NIR-II images displayed that the vascular system was clearly visualized in mice 30 minutes after injection. The signal-to-background ratios (SBRs) were measured by plotting cross-sectional intensity profiles of the blood vessels imaged in NIR-II window with IR-820-HSA. Moreover, using the Gaussian-fitted full width at half maximum (FWHM) of the cross-sectional intensity profiles of the blood vessels, FWHM of the corresponding vessel in the NIR-II window was obtained.

For the right low limb blood vessel in FIG. 3B, the adjacent artery and vein were distinguished in the middle part of the limb, with the SBR of 2.05, 2.25 and FWHM of 406 μm, 633 μm (FIG. 3D), respectively. The proximal end of the blood vessel showed higher fluorescence intensity with SNR of 2.93 (FIG. 3D). Small blood vessels were visible in the abdomen (FIG. 3C). Multiple parallel blood vessels were well displayed, with the highest SBR of 2.4 and FWHM of 246 μm (FIG. 3E). Clear blood vessels images were still available for 30 mins, indicating that IR-820-HSA had good circulation retention time in vivo. The SNR and FWHM of blood vessel showed the imaging quality of IR-820-HSA was comparable to that of the dedicated NIR-II agent (He et al., (2019) Nano Lett.; Shou et al., (2017) Adv. Funct. Mater. 27). NIR-II imaging of lymphatic system: To identify the lymphatic system including lymphatic vessels and lymph nodes under in vivo NIR-II imaging, heated IR-820-HSA was injected subcutaneously at the right-side footpad and middle tail of C57BL/6 mice, and unbound IR-820 was injected simultaneously at the left side as a comparison, with the mice in the prone position. A gentle massage was performed on the injection site for faster diffusion into the lymphatic system. The popliteal sciatic lymph nodes connected with lymphatic vessels were identified after probe injection into each side of the footpad. However, the fluorescence intensity on the right side was more evident (FIG. 4A). The right inguinal lymph node and connected lymphatic vessels were sequentially observed after the probe was subcutaneously injected into the middle of the tail.

Different afferent and efferent lymphatic vessels were also distinguished (FIGS. 4A-4C) (Harrell et al., (2008) J. Immunol. Methods 332: 170-174), but the identification of the inguinal lymph node in the left side failed under IR-820 injection. The resolution comparison of lymphatic vessels and lymph nodes imaged by IR-820 and IR-820-HSA was made using the SBRs and Gaussian-fitted FWHM.

As shown in FIG. 4D, compared with unbound IR-820, lymphatic vessels visualized in the NIR-II window with IR-820-HSA demonstrated significant enhanced feature sharpness and increased SBR and FWHM (4.77 and 551 μm vs. 1.33 and unmeasurable). These results illustrate the advantages of IR-820-HSA NIR-II imaging compared to unbound IR-820. The SBRs and FWHM of lymphatic vessels were also measured in lateral and supine position imaging (1.94 and 472 μm, 2.33 and 420 μm, respectively). IR-820-HSA as a probe, therefore, can be advantageous for studying a real time lymphatic drainage map in living subjects, and for quantitative assessment of lymphatic trafficking and function by in vivo imaging (Yamaji et al., (2018) Sci. Rep. 8: 5921; Proulx et al., (2017) JCI Insight 2: e90861).

### NIR-II Imaging for Assessment of Tumor Blood Supply Network:

To visualize the blood supply network in a tumor, NIR-II images were captured after injection of heated IR-820-HSA into the tail vein of tumor-bearing nude mice. The major blood vessel supplying the tumor accompanied with the branches and capillaries network to feed the tumor were observed in different long pass filters (FIGS. 5A and 5B). The original main blood supply vessels are shown in FIG. 5D and a week later in FIG. 5E, the results being verified after dissection, as shown in FIG. 5F.

The changes displayed in these two images showed the process of nutrient vascular thickening and twisting. The resolutions of the tumor blood vessels under different wavelength filters of 1000LP and 1200LP were compared using SBR and Gaussian FWHM. SBR and FWHM increased from 1.4 to 1.8 and from 656 μm to 441 μm, respectively (FIG. 5C). A tumor blood supply network with gradual change was clearly observed in NIR-II images, illustrating that fluorescence-visible monitoring is possible for tumor blood vessels treatment.

### NIR-II and PA Imaging of Tumor:

The feasibility of NIR II fluorescence imaging of the tumor with IR-820-HSA as the probe was investigated using BALB/c nude mice bearing subcutaneous 134B osteosarcoma tumors. After injection of IR-820-HSA, the mice were imaged by NIR II imaging and subsequent PA imaging under 808 nm excitation at multiple time points from 1 h to 48 h.

The tumor was displayed (FIGS. 6A and 6B) in NIR II fluorescence and photoacoustic imaging. The NIR II fluorescence signal in the tumor reached its maximum at about 18 h after injection. As shown in FIG. 6C, the distribution of IR-820-HSA in the tumor reached its peak at 18 h then dissipated. The tumor-to-background (T/B) ratios were calculated with the fluorescence intensity of hip site used as background signal. The T/B ratio reached its peak also at 18 h.

Ex vivo NIR II fluorescence imaging of the dissected tumor and organs obtained from tumor-bearing mice 8 h after injection were used to further assess the biodistribution of probe (FIG. 6D). Other than high NIR II fluorescence intensity observed in the tumor, there was accumulation of the probe in the liver, indicating that the probe was eliminated from the animal body by the digestive system (FIG. 6E). 7 days after imaging, the mice were killed and dissected. The H&E stained images of major organ slices (heart, lung, liver, spleen, kidney and tumor) were collected and no abnormality was observed. After coating with HSA, IR-820 successfully imaged the tumor with vasculature leakiness combined with poorly formed lymphatic drainage. The enhanced permeability and retention (EPR) effect is thought to be responsible for the preferential tumor accumulation of the IR-820-HSA macromolecule (Matsumura & Maeda (1986) Cancer Res. 46: 6387-6392).

### NIR-II Image-Guided Tumor Resection:

143B osteosarcoma tumor resections were performed under the guidance of NIR-II imaging after the IR-820-HSA imaged mouse was anesthetized. In NIR-II imaging (FIG. 7), tumor could be clearly recognized on the left shoulder where tumor cells had been injected before surgery. After incision of skin, the tumor was exposed with clear delineation with a tumor-to-background ratio of 15 between the tumor and the adjacent tissue. Under the guidance of dynamic NIR-II imaging, a resection was performed after the tumor had been completely removed, and no residue was found in NIR-II imaging.

After mixing the photoacoustic dye with HSA at a molar ratio of 1:2, and heating for 10 min at a temperature of 60° C., fluorescence in biological NIR-II window of the IR-820-HSA organic complex was increased by 20-fold. The complex exhibits a weak absorbance shoulder at 765 nm, and a strong peak at 835 nm, with a red-shift of 75 nm and 25 nm respectively. In vivo NIR-II imaging with IR-820-HSA can non-invasively and dynamically visualize and monitor physiological and pathological conditions of the vascular and lymphatic drainage systems, tumor-bearing mice, and even provide for image-guided tumor resections with high spatial and temporal resolution. Most advantageously, IR-820-HSA, as a multifunctional dual-modal imaging probe can integrate the benefits NIR-II fluorescence and photoacoustic imaging, and also as a laser-activated photoabsorber can lead to photothermal therapy, with the goal of providing diagnosis and treatment of cancer.

One aspect of the disclosure, therefore, encompasses embodiments of a probe comprising a fluorescent dye and a human serum albumin molecule, or fragment thereof, wherein the fluorescent dye is attached to the human serum albumin molecule, and wherein the fluorescent dye can have a greater near-infrared fluorescence intensity than when not in contact with the human serum albumin molecule.

In some embodiments of this aspect of the disclosure, the fluorescent dye can also be a photoacoustic emitter.

In some embodiments of this aspect of the disclosure, the fluorescent dye can be a cyanine dye.

In some embodiments of this aspect of the disclosure, the fluorescent dye can be IR-820. In some embodiments of this aspect of the disclosure, the dye can be attached to the human serum albumin, or fragment thereof, by at least one electrostatic bond, at least one covalent bond, or a combination thereof.

In some embodiments of this aspect of the disclosure, the dye can be conjugated to the human serum albumin, or fragment thereof, by at least one covalent bond.

In some embodiments of this aspect of the disclosure, the probe can be admixed with a pharmaceutically acceptable carrier.

Another aspect of the disclosure encompasses embodiments of a method of enhancing the NIR-II emission of a fluorescent dye, the method comprising synthesizing a composition comprising a fluorescent dye attached to a human serum albumin molecule, or fragment thereof, and wherein the fluorescent dye can have a greater near-infrared fluorescence than when not in contact with the human serum albumin molecule, the method comprising mixing aqueous solutions of the fluorescent dye and the human serum albumin, or fragment thereof, wherein the fluorescent dye and the human serum albumin, or fragment thereof, can have a molar ratio selected from the range of about 10:1 to about 1:10.

In some embodiments of this aspect of the disclosure, the fluorescent dye and the human serum albumin, or fragment thereof, can have a molar ratio of about 1:1.

In some embodiments of this aspect of the disclosure, the fluorescent dye can be a cyanine dye.

In some embodiments of this aspect of the disclosure, the fluorescent dye can be IR-820.

Still another aspect of the disclosure encompasses embodiments of a method of imaging a tissue or organ in an animal or human subject, the method comprising the steps of administering to an animal or human subject an amount of a probe, wherein the probe can comprise a fluorescent dye attached to a human serum albumin molecule, or fragment thereof, and wherein the fluorescent dye has a greater near-infrared fluorescence intensity than when not in contact with the human serum albumin molecule, generating a fluorescent signal from the probe by irradiating the exterior surface or a tissue of the subject with an excitation radiation specific for the fluorescent dye, detecting the emitted fluorescence and generating an image of the fluorescence relative to the body of the subject.

In some embodiments of this aspect of the disclosure, the method can further comprise irradiating the subject with a laser energy capable of generating a photoacoustic signal from the administered probe, detecting the photoacoustic signal emitted by the irradiated probe and generating an image of the fluorescence relative to the body of the subject.

In some embodiments of this aspect of the disclosure, the probe can be concentrated in a tumor of the subject, and wherein the photoacoustic energy generated from the probe can reduce at least one of the proliferation or the viability of the tumor.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLE

### Example 1

### Preparation of IR-820-HSA Complex:

IR-820 (500 μM) and HSA (500 μM) were first dissolved in deionized (DI) water, respectively. To prepare IR-820-HSA complex (10 μM) for optical characterization, IR-820 (10 μl) and HSA (20 μl) solution were then mixed into DI water to a final volume of 500 μl in an Eppendorf tube. Vortex the solution to mix evenly. In comparation with heated treatment, the solutions were put in water baths for 10 min at 60° C. for preparation of heated complex. If not used within a few hours, store the complex solution at 4° C. for long-term storage.

### Example 2

### Molecular Modeling:

SybyI-X software (Tripos Inc., MO) was used to prepare IR-820 and the crystal structure of HSA (obtained from RCSB Protein Data Bank, PDB ID: 1 h9z) before docking. A Surflex Dock package was utilized to carry out the molecular docking, with the parameters as follows: additional starting conformation per molecule: 20; Angstroms to expand search grid: 6; maximum conformation per fragment: 20; maximum number of rotatable bonds per molecule: 100.

### Example 3

### Spectral Characterization and Stability of IR-820-HSA:

Absorbance spectra of IR-820 and complexes were taken on an ultraviolet-visible-NIR Cary 60 spectrometer (Agilent Technologies) with background correction. The NIR-II fluorescence emission spectrum was captured on a home-built spectroscopy set-up by exciting IR-820 and complexes with an 808 nm laser diode with a power output of 100 mW. The excitation laser was filtered with a combination of 900 nm short-pass filters. Samples were added to a 1 cm path-length cuvette and a 1000 nm long-pass filter (Thorlabs) was used to reject the incident excitation laser light. The emitted fluorescence was collected by a spectrometer coupled to a cooled InGaAs detector array (Princeton Instruments, NIR vana: 640).

To investigate the photostability of IR-820-HSA complex in aqueous solution, fluorescence intensity of IR-820-HSA (10 μM), IR-820-HSA heated (10 μM) complex and free IR-820 (10 μM) in DI water were measured when exposed to continuous illumination at 808 nm for 20 minutes. After normalized by dividing the fluorescence intensity of each time point by the fluorescence intensity at t=0, the photobleaching curves were made. To study the stability of IR-820-HSA complex in PBS and FBS, IR-820-HSA (50 μM, 200 μl) and IR-820-HSA heated complex (50 μM, 200 μl) were added to PBS or 10% FBS (800 μl), respectively. Compared with IR-820-HSA in DI water, the fluorescence intensity (10 ms, 1000 LP) change was recorded at different time points up to 24 hours, then a normalized time fluorescence intensity curve was carried out.

### Example 4

### Cytotoxicity of IR-820-HSA:

The potential cytotoxicity of IR-820-HSA on normal cells NIH3T3 were examined using a standard MTT (Sigma-Aldrich, St. Louis, USA) assay. NIH3T3 cells (5×10³ cells per well) were seeded in 96-well plates in DMEM with 10% FBS. After 24 h, the medium of 96-well plates was replaced with 100 μl of medium containing different concentrations of IR-820-HSA (0 μM, 6.25 μM, 12.5 μM, 25 μM, 50 μM, 100 μM and 200 μM) and incubated for an additional 48 h. After that, MTT (10 μl, 0.5 mg/ml) solution was added to each well and incubated for 4 h at 37° C. The residues were lysed with 200 μl of dimethyl sulfoxide after removing the supernatant. Then a standard MTT method was performed for measuring the cell viability with a Bio-Rad microplate reader. The relative cell viability (%) was calculated by (Aₛₐₘₚₗₑ/A_{blank})×100%. All samples were used in triplicate, and the related experiments were all replicated three times.

### Example 5

### Cell Line and Animal Handling:

U87MG human glioma, 143B human bone osteosarcoma, and mouse embryonic fibroblast NIH/3T3 cell lines were obtained from the American Type Culture Collection and cultured in high-glucose Dulbecco's modified Eagle medium (DMEM) containing 10% FBS, 100 IU/mL penicillin, and 100 μg/mL streptomycin at 37° C. with 5% CO₂ atmosphere. Eight-week-old female C57BL/6 mice and NU/NU mice were used. About 2×10⁶ U87MG or 143B cells in DMEM media (no FBS) were hypodermically injected into the left shoulder of the nude mice.

Before imaging, all mice were anesthetized in a rodent anesthesia machine with 2 L/min O² gas mixed with 3% Soflurane. C57BL/6 mice were used for the vascular and lymphatic system NIR-II imaging. For the lymphatic imaging, the dyes (IR-820 through left side, IR-820-HSA through right side) were injected subcutaneously into the footpad (50 μl, 50 μM) and middle of the tail (20 μl, 50 μM) with the mice in the prone position on the imaging platform. A gentle massage was performed at the injection site to increase the speed of diffusion into the lymphatic system. For the vascular system and tumor imaging, IR-820-HSA complex (100 μl, 100 μM) and PBS (100 μl) was mixed and then injected by tail vein with the mice in the supine position under anesthesia. All groups within the study contained n=3 mice.

### Example 6

### In Vivo Animal NIR II Imaging:

All NIR-II images were collected on a two-dimensional InGaAs array NIR-II system (Princeton Instruments). The excitation was provided by an 808 nm diode laser through an optical fiber and collimator. Fluorescence emission was collected with 1000 nm or more than 1000 nm long-pass filters (Thorlabs). A lens set was used to obtain tunable magnifications ranging from 1× (whole body) to 2.5× (high magnification) by changing the relative position of two NIR achromats (75 mm and 200 mm, Thorlabs). Image J software were used for analyzing the images.

### Example 7

### In Vivo Animal Photoacoustic Imaging:

The photoacoustic signals were recorded using a Nexus 128 photoacoustic instrument (Endra Inc.) with a series of laser wavelengths in the range of 680-950 nm using a continuous rotation mode (with a scan time of 12 s per wavelength, 240 views, 1 pulse/view). The spatial resolution of PA imaging was limited to 280 μm.

The PA data was reconstructed in volumes of 256×256×256 with 0.1×0.1×0.1 mm voxels. The system was equipped with a tunable nanosecond pulsed laser (7 ns pulses, 20 Hz pulse repetition frequency, wavelength-dependent laser power density, about 4 to about 7 mJ/pulse on the animal surface) and 128 unfocused ultrasound transducers (with 5 MHz center frequency and 3 mm diameter) arranged in a hemispherical bowl filled with water (temperature is set to 38° C.). The imaging data was analyzed using Amide's a Medical Image Data Examiner (AMIDE and Osirix software (Pixmeo SARL).

### Example 8

### H&E Staining:

The obtained major organs were fixed with 4% paraformaldehyde overnight. Afterwards, these organs were embedded in optimal cutting temperature (OCT) compound (TISSUE-TEK®, Sakura Finetek, USA), sectioned into 8 μm slices in the cryostat at −20° C. with a microtome and transferred onto microscope slides for hematoxylin and eosin (H&E) using SHANDON® rapid Chrome kit (Thermo Scientific, USA). The stained sections were imaged under NanoZoomer 2.0RS.

### Example 9

### Statistical Analysis:

The fluorescence measurement was performed to quantify NIR-II fluorescence signal intensity through the Image J software (National Institutes of Health, Bethesda, Md.). The line graphs and Gaussian-fitted FWHM were analysis by origin 8.5 (OriginLab Corporation, Northampton, Mass.).

What is claimed:

1. A probe comprising a fluorescent dye and a human serum albumin molecule, or fragment thereof, wherein the fluorescent dye is attached to the human serum albumin molecule, and wherein the fluorescent dye has a greater near-infrared fluorescence intensity than when not in contact with the human serum albumin molecule.

2. The probe of claim 1, wherein the fluorescent dye is also a photoacoustic emitter.

3. The probe of claim 1, wherein the fluorescent dye is a cyanine dye.

4. The probe of claim 3, wherein the fluorescent dye is IR-820.

5. The probe of claim 1, wherein the dye is attached to the human serum albumin, or fragment thereof, by at least one electrostatic bond, at least one covalent bond, or a combination thereof.

6. The probe of claim 1, wherein the dye is conjugated to the human serum albumin, or fragment thereof, by at least one covalent bond.

7. The probe of claim 1, wherein the probe is admixed with a pharmaceutically acceptable carrier.

8. A method of enhancing the NIR-II emission of a fluorescent dye, the method comprising synthesizing a composition comprising a fluorescent dye attached to a human serum albumin molecule, or fragment thereof, and wherein the fluorescent dye has a greater near-infrared fluorescence than when not in contact with the human serum albumin molecule, the method comprising mixing aqueous solutions of the fluorescent dye and the human serum albumin, or fragment thereof, wherein the fluorescent dye and the human serum albumin, or fragment thereof, have a molar ratio selected from the range of about 10:1 to about 1:10.

9. The method of claim 8, wherein the fluorescent dye and the human serum albumin, or fragment thereof, have a molar ratio of about 1:1.

10. The method of claim 8, wherein the fluorescent dye is a cyanine dye.

11. The method of claim 8, wherein the fluorescent dye is IR-820.

12. A method of imaging a tissue or organ in an animal or human subject, the method comprising the steps of administering to an animal or human subject an amount of a probe, wherein the probe comprises a fluorescent dye attached to a human serum albumin molecule, or fragment thereof, and wherein the fluorescent dye has a greater near-infrared fluorescence intensity than when not in contact with the human serum albumin molecule, generating a fluorescent signal from the probe by irradiating the exterior surface or a tissue of the subject with an excitation radiation specific for the fluorescent dye, detecting the emitted fluorescence and generating an image of the fluorescence relative to the body of the subject.

13. The method of claim 12, further comprising irradiating the subject with a laser energy capable of generating a photoacoustic signal from the administered probe, detecting the photoacoustic signal emitted by the irradiated probe and generating an image of the fluorescence relative to the body of the subject.

14. The method of claim 13, wherein the probe is concentrated in a tumor of the subject, and wherein the photoacoustic energy generated from the probe reduces at least one of the proliferation or the viability of the tumor.
